(19) United States
(12) Patent Application Publication
(10) Pub. No.: US 2026/0167922 A1
(43) Pub. Date: Jun. 18, 2026

(54) METHODS FOR LARGE-SCALE PRODUCTION OF MAGNETOSOMES

(71) Applicant: SUPERBREWED FOOD INC., New Castle, DE (US)

(72) Inventors: Biniam MARU, Bear, DE (US); Bryan P. TRACY, Wilmington, DE (US); Hadar GILARY, Kibbutz Magal (IL); Aharon EYAL, Jerusalem (IL); Carrissa WIEDEL, Bel Air, MD (US); John Randall PHILLIPS, Middletown, DE (US)

(21) Appl. No.: 19/127,111

(22) PCT Filed: Nov. 6, 2023
(86) PCT No.: PCT/IB2023/061149

§ 371 Date: May 4, 2025

Related U.S. Application Data
(60) Provisional application No. 63/422,434, filed on Nov. 4, 2022.

(51) Int. Cl. C12N 1/20

(52) U.S. Cl. CPC C12N 1/20

(57) ABSTRACT

Provided is a method for large-scale production of magnetosomes, the method comprising maintaining in a fermentation vessel under continuous fermentation conditions a fermentation broth comprising magnetotactic bacteria for a duration of at least 100 hours and during said maintaining, providing to said fermentation broth at least one feed composition comprising iron ions and molecules and/or ions of at least 4 selected from the group consisting of lactic acid, ammonia, magnesium, potassium, thiamine, calcium and combinations thereof at an average feed rate of between 10% and 150% of the volume of the fermentation vessel per day; adding oxygen to said fermentation broth in said fermentation vessel; and removing a portion of said fermentation broth comprising said magnetotactic bacteria at an average rate of between 10% and 150% of the fermentation vessel volume per day.

### CROSS-REFERENCE TO RELATED APPLICATION

The present application gains priority from U.S. Provisional Application No. 63/422,434 filed Nov. 4, 2022 which is incorporated by reference as if fully set-forth herein.

### FIELD OF THE INVENTION

The present invention, in at least some embodiments, relates to production of magnetosomes, and in particular to large-scale production of magnetosomes from magnetotactic bacteria.

### BACKGROUND OF THE INVENTION

Biomineralization is the process by which living organisms produce minerals. It is known to use magnetotactic bacteria (MTB) in order to biomineralize crystalline single domain magnetic oxides and sulfides, known as magnetosomes (MGS).

MGS have several advantages compared to synthetic magnetic nanoparticles, such as uniform size high crystalline structures with a narrow size distribution of 40-50 nm, high intrinsic saturation magnetization, and lower toxicity. Until recently, application development has been limited due to the production scale, downstream processing, and the ability to tailor MTB to produce particles that have desired properties.

### SUMMARY OF THE INVENTION

The present inventors have developed an improved method for production of magnetosomes. The method enables continuous, stable chemostat fermentation over a longtime period, providing greater control over particle size, composition, and magnetization as compared to known methods.

The method of the present invention provides improved upstream media and process development, as well as downstream processing to improve the recovery of the high-value magnetic particles. Producing MGS at this scale has allowed the inventors to thoroughly evaluate these nanoparticles for a range of functional composite applications, including medical uses, radio-frequency waveguide and electromagnetic coatings. The recovery process enables the magnetic particles to be freed from the bulk cell mass.

Small-angle X-ray scattering, dynamic light scattering, and electron microscopy with energy filtering may be used to thoroughly characterize the structure, crystallinity, and size of different batches of MGS as different timepoints during the controlled continuous fermentation.

According to an aspect of some embodiments of the present invention, there is provided a method for large-scale production of magnetosomes, the method comprising maintaining in a fermentation vessel, under continuous fermentation conditions, a fermentation broth comprising magnetotactic bacteria for a duration of at least 100 hours, and during the maintaining (a) providing to the fermentation broth in the fermentation vessel at least one feed composition comprising iron ions and molecules and/or ions of at least 4 selected from the group consisting of lactic acid, ammonia, magnesium, potassium, thiamine, calcium and combinations thereof, at an average feed rate of between 10% and 150% of the volume of the fermentation vessel per day; (b) adding oxygen to the fermentation broth in the fermentation vessel; and (c) removing a portion of the fermentation broth comprising the magnetotactic bacteria at an average rate of between 10% and 150% of the fermentation vessel volume per day.

### DETAILED DESCRIPTION OF THE INVENTION

The present invention provides a method for the large-scale production of magnetosomes from magnetotactic bacteria.

The particulars shown herein are by way of example and for purposes of illustrative discussion of the various embodiments of the present invention only and are presented in the cause of providing what is believed to be the most useful and readily understood description of the principles and conceptual aspects of the invention. In this regard, no attempt is made to show details of the invention in more detail than is necessary for a fundamental understanding of the invention, the description making apparent to those skilled in the art how the several forms of the invention may be embodied in practice.

The present invention will now be described by reference to more detailed embodiments. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention.

As used herein, the term “large-scale production” refers to production of at least 1 g/L per day of magnetosome on dry basis.

As used herein, the term “magnetotactic bacteria” refers to prokaryotic bacteria having organelles known as magnetosomes that contain magnetic crystals, such as crystals of magnetite or greigite, which enable the bacteria to orient themselves along the magnetic field lines of Earth's magnetic field.

As used herein, the term “under continuous fermentation conditions”, refers to conditions wherein feed composition is added and fermentation broth containing magnetotactic bacteria is removed throughout the production process once steady state has been achieved. The term “throughout the production process” as used herein is intended to encompass a single operation or multiple operations.

As used herein, the term “a portion” refers to an amount of less than 100%.

As used herein, the term “molecules and/or ions” refers to uncharged molecules and/or to atoms or molecules having a net electrical charge.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Every numerical range given throughout this specification will include every narrower numerical range that falls within such broader numerical range, as if such narrower numerical ranges were all expressly written herein.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

As used herein, when a numerical value is preceded by the term “about”, the term “about” is intended to indicate +/−10% of that value.

As used herein, the terms “comprising”, “including”, “having” and grammatical variants thereof are to be taken as specifying the stated features, integers, steps or components but do not preclude the addition of one or more additional features, integers, steps, components or groups thereof. These terms encompass the terms “consisting of” and “consisting essentially of”.

Additional advantages of the invention will be set forth in part in the description which follows, and in part will be obvious from the description, or may be learned by practice of the invention. It is to be understood that both the foregoing general description and the following detailed description are exemplary and explanatory only and are not restrictive of the invention, as claimed.

According to an aspect of some embodiments of the present invention, there is provided a method for large-scale production of magnetosomes, the method comprising maintaining in a fermentation vessel under continuous fermentation conditions a fermentation broth comprising magnetotactic bacteria for a duration of at least 100 hours and during said maintaining, (a) providing to the fermentation broth at least one feed composition comprising iron ions and molecules and/or ions of at least 4 selected from the group consisting of lactic acid, ammonia, magnesium, potassium, thiamine, calcium and combinations thereof, at an average feed rate of between 10% and 150% of the volume of the fermentation vessel per day; (b) adding oxygen to the fermentation broth; and (c) removing a portion of the fermentation broth comprising the magnetotactic bacteria at an average rate of between 10% and 150% of the fermentation vessel volume per day.

According to some embodiments, the fermentation vessel comprises a bioreactor system, optionally configured to maintain a level of dissolved oxygen at less than 20 ppb, and to maintain the flow of fermentation broth.

According to some embodiments, oxygen is added during at least 1%, at least 2%, at least 5%, at least 10%, at least 15%, at least 20%, at least 25%, at least 30%, at least 35%, at least 45%, at least 50%, at least 55%, at least 60%, at least 65%, at least 70%, at least 75%, at least 80%, at least 85%, at least 90%, at least 95% or about 100% of a total duration of time during which the continuously repeating of steps (ii) and (iii) is conducted.

According to some embodiments, the oxygen is added at a concentration in a range between about 0.001% and about 21% is added to the modified fermentation broth at an oxygen feed rate in a range between about 0.001 mL/min and about 0.5 L/min.

According to some embodiments, oxygen is added in an overlay or sparging mode.

According to some embodiments, the fermentation broth comprises any suitable liquid fermentation medium which provides at least a carbon source, a nitrogen source, salts, water and micronutrients in amounts sufficient to meet the nutritional requirements of the magnetotactic bacteria. Examples of suitable media include, without limitation, magnetospirillum medium 380 from Liebniz Institute, Germany and magnetospirillum medium 1653 from ATCC, Virginia, United States. According to some embodiments, the medium is a minimal growth medium.

According to some embodiments, the at least one feed composition comprises iron ions selected from the group consisting of ferric ions, ferrous ions and combinations thereof.

According to some embodiments, the at least one feed composition comprises molecules and/or ions of 4, 5 or all 6 selected from the group consisting of lactic acid, ammonia, magnesium, potassium, thiamine and calcium.

According to some embodiments, the at least one feed composition comprises a single composition comprising iron ions and at molecules and/or ions of least 4 selected from the group consisting of lactic acid, ammonia, magnesium, potassium, thiamine, calcium and combinations thereof.

According to some embodiments, the at least one feed composition comprises at least two feed compositions, wherein at least a first feed composition comprises iron ions and at least a second feed composition comprises molecules and/or ions of at least 4 selected from the group consisting of lactic acid, ammonia, magnesium, potassium, thiamine, calcium and combinations thereof, wherein the first feed composition is different from the second feed composition.

According to some embodiments, each of the iron ions and the molecules and/or ions of at least 4 selected from the group consisting of lactic acid, ammonia, magnesium, potassium, thiamine and calcium is provided in a separate feed composition. According to some embodiments, the iron ions are provided in a first feed composition together with one or more of the molecules and/or ions of lactic acid, ammonia, magnesium, potassium, thiamine and calcium; and the molecules and/or ions of the remaining number of the at least 4 selected from the group consisting of lactic acid, ammonia, magnesium, potassium, thiamine and calcium are provided in one or more separate feed compositions.

According to some embodiments, the at least one feed composition is provided to the fermentation broth at an average feed rate of about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 105%, about 110%, about 115%, about 120%, about 125%, about 130%, about 135%, about 140%, about 145% or about 150% of the volume of the fermentation vessel per day.

According to some embodiments, the portion of the fermentation broth comprising the magnetotactic bacteria is removed at an average rate of about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 105%, about 110%, about 115%, about 120%, about 125%, about 130%, about 135%, about 140%, about 145% or about 150% of the fermentation vessel volume per day.

According to some embodiments, the providing of the at least one feed composition to the fermentation broth is carried out substantially continuously during maintaining of the fermentation broth in the fermentation vessel.

According to some embodiments, the providing of the at least one feed composition to the fermentation broth is carried out intermittently during maintaining of the fermentation broth in the fermentation vessel.

According to some embodiments, the removing of a portion of the fermentation broth is carried out substantially continuously during maintaining of the fermentation broth in the fermentation vessel.

According to some embodiments, the removing of a portion of the fermentation broth is carried out intermittently during maintaining of the fermentation broth in the fermentation vessel.

According to some embodiments, oxygen is added to the modified fermentation broth at a concentration of about 0.001%, about 0.005%, about 0.01%, about 0.05%, about 0.1%, about 0.5%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20% or about 21%.

According to some embodiments, oxygen is added to the modified fermentation broth at an oxygen feed rate or about 0.001 mL/min, about 0.005 mL/min, about 0.01 mL/min, about 0.05 mL/min, about 0.1 mL min, or about 0.5 mL/min.

According to some embodiments, an optical density (OD) of the fermentation broth is in a range between 1 and 30, such as 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30.

According to some embodiments, the method further comprises maintaining the OD in the range during at least a portion of the duration, such as at least 1%, at least 2%, at least 5%, at least 10%, at least 15%, at least 20%, at least 25%, at least 30%, at least 35%, at least 45%, at least 50%, at least 55%, at least 60%, at least 65%, at least 70%, at least 75%, at least 80%, at least 85%, at least 90%, at least 95% or about 100% of the duration. According to some such embodiments, maintaining comprises monitoring the OD, and when the OD is less than 1, increasing the oxygen feed rate by at least about 5%, such as 5%, 10%, 15%, 20%, 25%, 30%, 35%, 40%, 45%, 50%, 55%, 60%, 65%, 70%, 75%, 80%, 85%, 90%, 95%, 100% or even greater than 100% of the oxygen feed rate at the time of monitoring.

According to some embodiments, a coefficient of magnetism (Cmag) of the fermentation broth is in a range between 1.2 and 3, such as 1.2, 1.3. 1.4, 1.5, 1.6, 1.7, 1.8, 1.9, 2.0, 2.1, 2.2, 2.3, 2.4, 2.5, 2.6, 2.7, 2.8, 2.9, 3.0 or 3.1.

According to some embodiments, the method further comprises maintaining the Cmag in the range during at least a portion of the duration, such as at least 1%, at least 2%, at least 5%, at least 10%, at least 15%, at least 20%, at least 25%, at least 30%, at least 35%, at least 45%, at least 50%, at least 55%, at least 60%, at least 65%, at least 70%, at least 75%, at least 80%, at least 85%, at least 90%, at least 95% or about 100% of the duration. According to some such embodiments, maintaining comprises monitoring the Cmag, and when the Cmag is less than 2, decreasing the oxygen feed rate by at least about 1%, such as 1%, 2%, 5%, 10%, 15%, 20%, 25%, 30%, 35%, 40%, 45%, 50%, 55%, 60%, 65%, 70%, 75%, 80%, 85%, 90%, 95%, 100% or even greater than 100% of the oxygen feed rate at the time of monitoring.

According to some embodiments, a temperature of the fermentation broth is in a range between 25° C. and 33° C., such as 25° C., 26° C., 27° C., 28° C., 29° C., 30° C., 31° C., 32° C. or 33° C.

According to some embodiments, the method further comprises maintaining the temperature in the range during at least a portion of the duration, such as at least 1%, at least 2%, at least 5%, at least 10%, at least 15%, at least 20%, at least 25%, at least 30%, at least 35%, at least 45%, at least 50%, at least 55%, at least 60%, at least 65%, at least 70%, at least 75%, at least 80%, at least 85%, at least 90%, at least 95% or about 100% of the duration. According to some such embodiments, maintaining comprises, monitoring the temperature and a) applying heat to said fermentation broth when the temperature is less than 25° C.; and b) cooling the fermentation broth when the temperature is greater than 33° C.

According to some embodiments, the fermentation broth comprises oxygen at a concentration in a range between about 0.1% and about 5% of, such as about 0.1%, about 0.5%, about 1.0%, about 1.5%, about 2.0%, about 2.5%, about 3.0%, about 3.5%, about 4.0%, about 4.5% or about 5.0% of saturation.

According to some embodiments, the method further comprises maintaining the oxygen concentration in the range during at least a portion of the duration, such as at least 1%, at least 2%, at least 5%, at least 10%, at least 15%, at least 20%, at least 25%, at least 30%, at least 35%, at least 45%, at least 50%, at least 55%, at least 60%, at least 65%, at least 70%, at least 75%, at least 80%, at least 85%, at least 90%, at least 95% or about 100% of the duration. According to some such embodiments, maintaining comprises monitoring the oxygen concentration and a) when the oxygen concentration is less than 0.1 of saturation, increasing the oxygen feed rate; and b) when the oxygen concentration is greater than 5% of saturation, reducing the oxygen feed rate.

According to some embodiments, a pH of the broth is in a range between 6.5 and 7.5, such as 6.5, 6.6, 6.7, 6.8, 6.9, 7.0, 7.1, 7.2, 7.3, 7.4, 7.5, 7.6 or 7.7.

According to some embodiments, the method further comprises maintaining the pH in the range during at least a portion of the duration, such as at least 1%, at least 2%, at least 5%, at least 10%, at least 15%, at least 20%, at least 25%, at least 30%, at least 35%, at least 45%, at least 50%, at least 55%, at least 60%, at least 65%, at least 70%, at least 75%, at least 80%, at least 85%, at least 90%, at least 95% or about 100% of a total duration of time during which the continuously repeating of steps (ii) and (iii) is conducted. According to some such embodiments, maintaining comprises monitoring the pH and a) adding an acid to the fermentation broth when the pH is greater than 7.5; and b) adding a base to the fermentation broth when the pH is less than 6.5. According to some such embodiments, a pH of the feed composition is from about 7.0 to about 7.5, such as about 7.0, about 7.1, about 7.2, about 7.3, about 7.4 or about 7.5.

According to some embodiments, a total concentration of iron ions in the feed composition is in a range between about 1 μM and about 100 μM, such as about 1 μM, about 5 μM, about 10 μM, about 15 μM, about 20 μM, about 25 μM, about 30 μM, about 35 μM, about 40 μM, about 45 μM, about 50 μM, about 55 μM, about 60 μM, about 65 μM, about 70 μM, about 75 μM, about 80 μM, about 85 μM, about 90 μM or about 100 μM.

According to some embodiments, the iron ions comprise both ferrous ions and ferric ions at a ferric to ferrous weight/weight ratio in a range between about 0.01 and about 100, such as about 0.01, about 0.05, about 0.1, about 0.5, about 1.0, about 1.5, about 10, about 15, about 20, about 25, about 30, about 35, about 40, about 45, about 55, about 60, about 65, about 70, about 75, about 80, about 85, about 90, about 95 or about 100. The presence of both ferrous ions and ferric ions helps drive assembly of magnetite which is composed of both such ions.

According to some embodiments, the feed composition comprises lactic acid at a concentration in a range between about 1 mM and about 2000 mM, such as about 1 mM, about 5 mM, about 10 mM, about 20 mM, about 30 mM, about 40 mM, about 50 mM, about 60 mM, about 70 mM, about 80 mM, about 90 mM, about 100 mM, about 200 mM, about 300 mM, about 400 mM, about 500 mM, about 600 mM, about 700 mM, about 800 mM, about 900 mM, about 1000 mM, about 1100 mM, about 1200 mM, about 1300 mM, about 1400 mM, about 1500 mM, about 1600 mM, about 1700 mM, about 1800 mM, about 1900 mM or about 2000 mM.

According to some embodiments, the feed composition comprises ammonia at a concentration in a range between about 1 mM and about 1000 mM, such as about 1 mM, about 5 mM, about 10 mM, about 20 mM, about 30 mM, about 40 mM, about 50 mM, about 60 mM, about 70 mM, about 80 mM, about 90 mM, about 100 mM, about 200 mM, about 300 mM, about 400 mM, about 500 mM, about 600 mM, about 700 mM, about 800 mM, about 900 mM or about 1000 mM.

According to some embodiments, the feed composition comprises magnesium at a concentration in a range between about 0.01 mM and about 10 mM, such as about 0.01 mM, about 0.05 mM, about 0.1 mM, about 0.5 mM, about 1 mM, about 2 mM, about 3 mM, about 4 mM, about 5 mM, about 6 mM, about 7 mM, about 8 mM, about 9 mM or about 10 mM.

According to some embodiments, the feed composition comprises potassium at a concentration in a range between about 0.1 mM and about 20 mM, such as about 0.01 mM, about 0.05 mM, about 0.1 mM, about 0.5 mM, about 1 mM, about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM or about 20 mM.

According to some embodiments, the feed composition comprises thiamine at a concentration in a range between about 0 mM and about 5 mM, such as about 0 mM, about 0.5 mM, about 1.0 mM, about 1.5 mM, about 2 mM, about 2.5 mM, about 3 mM, about 3.5 mM, about 4 mM, about 4.5 mM or about 5 μM.

According to some embodiments, the feed composition comprises calcium at a concentration in a range between about 0.01 mM and about 5 mM, such as about 0.01 mM, about 0.05 mM, about 0.1 mM, about 0.5 mM, about 1 mM, about 1.5 mM, about 2 mM, about 2.5 mM, about 3 mM, about 3.5 mM, about 4 mM, about 4.5 mM or about 5 mM.

According to some embodiments, a total duration of time during which the continuously repeating steps (ii) and (iii) is conducted is at least 300 hours.

According to some embodiments, the magnetotactic bacterium is selected from the group consisting of Magnetospirillum, species Magnetospirillum magnetotacticum, Magnetospirillum gryphiswaldense Magnetospirillum. Magneticum, Magnetospirillum bellicus, Magnetospirillum caucaseum, Magnetospirillum marisnigri, Magnetospirillum moscoviense and combinations thereof.

According to an aspect of some embodiments of the present invention, there is provided at least one magnetosome produced by the method as disclosed herein. According to some such embodiments, the at least one magnetosome comprises at least 3 magnetosomes in the form of a chain.

According to an aspect of some embodiments of the present invention, there is provided a product comprising the magnetosome as disclosed herein.

According so some embodiments, the produce is selected from the group consisting of a biomaterial and a biomedical material, such as a biomedical material selected from the group consisting of a contrast agent for magnetic particle imaging, a material for use in targeted drug delivery, a material used for cancer treatment, a material for use in a medical diagnosis, a bioreagent, a material for use in cytophotometry and a material for use in bioremediation.

According to an aspect of some embodiments of the present invention, there is provided a method selected from the group consisting of an electronic application for signal capture, signal detection, signal amplification, bioconversion of immobilized to free cells, liquid filtration, 3D printing, ink Jet printing, wet electro spinning, an absorption method and a deposition method, comprising use of the product as disclosed herein.

According to some embodiments, the product is selected from the group consisting of a capacitor, a composite material and a conducting material.

1. A method for large-scale production of magnetosomes, the method comprising maintaining in a fermentation vessel under continuous fermentation conditions a fermentation broth comprising magnetotactic bacteria for a duration of at least 100 hours, and during said maintaining,
(a) providing to said fermentation broth at least one feed composition comprising iron ions and molecules and/or ions of at least 4 selected from the group consisting of lactic acid, ammonia, magnesium, potassium, thiamine, calcium and combinations thereof at an average feed rate of between 10% and 150% of the volume of the fermentation vessel per day;
(b) adding oxygen to said fermentation broth; and
(c) removing a portion of said fermentation broth comprising said magnetotactic bacteria at an average rate of between 10% and 150% of the fermentation vessel volume per day.

2. (canceled)

3. (canceled)

4. (canceled)

5. (canceled)

6. The method of claim 1, wherein an optical density (OD) of said fermentation broth is in a range between 1 and 30.

7. The method of claim 6, further comprising maintaining said OD in said range during at least a portion of said duration, wherein said maintaining comprises monitoring said OD and when said OD is less than 1, increasing said oxygen feed rate by at least 5% of the oxygen feed rate at the time of monitoring.

8. (canceled)

9. The method of claim 1, wherein a coefficient of magnetism (Cmag) of said fermentation broth is in a range between 1.2 and 3.

10. The method of claim 9, further comprising maintaining said Cmag in said range during at least a portion of said duration, wherein said maintaining comprises monitoring said Cmag and when said Cmag is less than 2, decreasing said oxygen feed rate by at least 1% of the oxygen feed rate at the time of monitoring.

11. (canceled)

12. The method of claim 1, wherein a temperature of said fermentation broth is in a range between 25° C. and 33° C.

13. (canceled)

14. The method of claim 1, wherein said fermentation broth comprises oxygen at a concentration in a range between 0.1% and 5% of saturation.

15. The method of claim 14, further comprising maintaining said oxygen concentration in said range during at least a portion of said duration, wherein said maintaining comprise monitoring said oxygen concentration and a) when said oxygen concentration is less than 0.1% of saturation, increasing said oxygen feed rate; and b) when said oxygen concentration is greater than 5% of saturation, reducing said oxygen feed rate.

16. (canceled)

17. The method of claim 1, wherein a pH of said broth is in a range between 6.5 and 7.5.

18. The method of claim 17, further comprising maintaining said pH in said range during at least a portion of said duration.

19. The method of claim 1, wherein a pH of said feed composition is from about 7.0 to about 7.5.

20. (canceled)

21. The method of claim 1, wherein said iron ions comprise both ferrous ions and ferric ions at a ferric to ferrous weight/weight ratio in a range between 0.01 and 100.

22. The method of claim 1, wherein said feed composition comprises lactic acid and/or lactate ions at a concentration in a range between 1 mM and 2000 mM.

23. The method of claim 1, wherein said feed composition comprises ammonia and/or ammonium ions at a concentration in a range between 1 mM and 100 mM.

24. The method of claim 1, wherein said feed composition comprises magnesium ions at a concentration in a range between 0.01 mM and 10 mM.

25. The method of claim 1, wherein said feed composition comprises potassium ions at a concentration in a range between 0.1 mM and 20 mM.

26. The method of claim 1, wherein said feed composition comprises thiamine at a concentration in a range between 0 mM and 5 mM.

27. The method of claim 1, wherein said feed composition comprises calcium ions at a concentration in a range between 0.01 mM and 5 mM.

28. The method of claim 1, wherein said duration is at least 300 hours.

29. The method of claim 1, wherein said magnetotactic bacterium is selected from the group consisting of Magnetospirillum, species Magnetospirillum magnetotacticum, Magnetospirillum. Gryphiswaldense Magnetospirillum. Magneticum, Magnetospirillum bellicus, Magnetospirillum caucaseum, Magnetospirillum marisnigri, and Magnetospirillum moscoviense.

30. (canceled)

31. (canceled)

32. (canceled)

33. (canceled)

34. (canceled)

35. (canceled)

36. (canceled)
